(19) United States
(12) Patent Application Publication
(10) Pub. No.: US 2020/0329988 A1
(43) Pub. Date: Oct. 22, 2020

(54) FUNCTIONAL NETWORK ANALYSIS SYSTEMS AND ANALYSIS METHOD FOR COMPLEX NETWORKS

(71) Applicant: FIRST INSTITUTE OF OCEANOGRAPHY, MINISTRY OF NATURAL RESOURCES, QINGDAO (CN)

(72) Inventors: JIARONG YEH, QINGDAO (CN); NORDEN E HUANG, QINGDAO (CN)

(21) Appl. No.: 16/851,109

(22) Filed: Apr. 17, 2020

(30) Foreign Application Priority Data
Apr. 22, 2019 (CN) 201910322863.3

(51) Int. Cl. A61B 5/048; A61B 5/00; A61B 5/04

(52) U.S. Cl. CPC A61B 5/048; A61B 5/4088; A61B 5/7246; A61B 5/742; A61B 5/04012

(57) ABSTRACT

A functional network analysis system for complex network provided by the present application, includea multi-signal-source measurement unit, a signal decomposition unitand a cross-frequency coupling analysis unit, wherein the multi-signal-source measurement unit, the signal decomposition unitand the cross-frequency coupling analysis unit are connected successively. A method using the functional network analysis system is more suitable for the decomposition of non-linear and non-stationary data in complex networks, and can reflect the dynamic properties of functional network links between different frequency bands in a complex system.

### CROSS REFERENCE TO RELATED APPLICATION

This application claims the priority benefit of Chinese application serial No. 201910322863.3, filed on Apr. 22, 2019, which is hereby incorporated herein by reference in its entirety.

### TECHNICAL FIELD

The present application belongs to the technical field of network analysis, and particularly relates to a functional network analysis system and analysis method for complex network.

### BACKGROUND OF THE PRESENT INVENTION

As technologies currently used to represent functional network connection relationship in the brain, clinical imaging system using functional magnetic resonance imaging (fMRI) is the first choice. This system uses blood oxygenation level dependent (BOLD) in the cerebral blood flow, or the diffusion-weighted magnetic resonance imaging (DWI or DW-MRI) or diffusion tensor images (DTIs) of water molecules to represent the functional activities in the brain. However, both DWI and DTI are signals with low temporal resolution, which cannot accurately reflect the functional mental activities of the brain caused by actual event stimulation. Furthermore, MRI examinations are expensive. Compared with MRI, Electroencephalogram (EEG) is convenient and non-invasive. It can measure high-frequency dynamic EEG activities in the whole brain to obtain non-linear and non-stationary EEG data. Low cost and high temporal resolution are the two major advantages of the EEG system. Cross-frequency phase-amplitude coupling (CF-PAC) analysis is a new data tool used to quantify the coupling relationship between the phase of low-frequency components and the amplitude of high-frequency components in one signal. The CF-PAC analysis usually uses a band-pass filter bank to capture signal components at different set bands.

### SUMMARY OF THE PRESENT INVENTION

An objection of the present application is to provide a functional network analysis system and method which can apply in complex network.

For this purpose, the present application provides the following technical solution:

afunctional network analysis system for complex network, wherein, comprising a multi-signal-source measurement unit, a signal decomposition unitand a cross-frequency coupling analysis unit, wherein the multi-signal-source measurement unit, the signal decomposition unitand the cross-frequency coupling analysis unit are connected successively;

the multi-signal-source measurement unit is configured to measure signal data from signal sources in a complex network and send the measured signal data of the signal sources to the signal decomposition unit;

the signal decomposition unit is configured to receive the measured signal data, decompose a plurality of different Mode Functions from the measured signal data by signal decomposition algorithms, and send the plurality of different Mode Functions to the cross-frequency coupling analysis unit; and

the cross-frequency coupling analysis unit is configured to receive the plurality of different Mode Functions, select a certain number of different Mode Functions from a same signal source or different signal sources, combine the selected different Mode Functions in pairs, quantify the coupling relationship between the phase of low-frequency Mode Function and the amplitude of high-frequency Mode Function in each combination, and arrange the quantified coupling relationship to generate a correlation coefficient matrix.

Optionally, the functional network analysis system for complex network further comprises a functional network analysis unit which is connected to the cross-frequency coupling analysis unit;

the functional network analysis unit is configured to receive the correlation coefficient matrix sent by the cross-frequency coupling analysis unit; obtain functional network attribute indicators based on Graph theory by using the correlation coefficient matrix as a calculation basis; and present, in the form of graph, the functional network connection relationship described by the functional network attribute indicators.

Optionally, the functional network analysis system for complex network further comprises a man-machine interaction device connected to the functional network analysis unit, the man-machine interaction device is configured to display a man-machine interaction interface to a user, receive a graph, which presents the functional network connection relationship described by functional network attribute indicators, sent by the functional network analysis unit, and display the graph on the man-machine interaction interface.

Optionally, the man-machine interaction device is further connected to the multi-signal-source measurement unit, and is configured to acquire signal source measurement instructions input by the user on the man-machine interaction interface and send the signal source measurement instructions to the multi-signal-source measurement unit, wherein the signal source measurement instructions comprise the number of signal sources and the kind of signal sources.

Optionally, the cross-frequency coupling analysis unit comprises a Mode Function selection module and a correlation coefficient matrix generation module;

the Mode Function selection module is configured to receive the plurality of different Mode Functions, and select n different Mode Functions from a same signal source or different signal sources;

the correlation coefficient matrix generation module is configured to combine the selected n different Mode Functions in pairs, quantify coupling relationship between the phase of low-frequency Mode Function and the amplitude of high-frequency Mode Function in each combination, and arrange the quantified coupling relationship to generate C(n,2) m×m correlation coefficient matrixes;

wherein, C(n,2) is obtained by permutation and combination, that is, C(n,2)=n*(n−1)/2, the value of C(n,2) represents the number of the correlation coefficient matrixes; n is the number of the selected different Mode Functions; m is the number of the signal sources; m×m indicates that the number of rows and number of columns of the correlation coefficient matrixes is m; each element in the correlation coefficient matrix is defined as the difference between the probability density distribution of the phase of the j^{th} Mode Function and the amplitude of the i^{th} Mode Function (for short, the probability density distribution of the low-frequency phase and the high-frequency amplitude) and the uniform probability distribution (that is, the probability density distribution of Uniform Distribution), and is used as a measurement indicator for the coupling relationship between the phase of a low-frequency Mode Function and the amplitude of a high-frequency Mode Function from a same signal source or different signal sources, where 1≤i≤n−1, i<j≤j and j is an integer greater than 1.

Optionally, the signal decomposition unit decomposes a plurality of different Intrinsic Mode Functions (IMFs) from the signal data of the signal sources by EMD, and sends the plurality of different IMFs to the cross-frequency coupling analysis unit; and

the cross-frequency coupling analysis unit is configured to receive the plurality of different IMFs, select a certain number of IMFs from a same signal source or different signal sources, combine the selected different IMFs in pairs, quantify coupling relationship between the phase of low-frequency IMF and the amplitude of high-frequency IMF in each combination, and arrange the quantified coupling relationship to generate a correlation coefficient matrix.

Optionally, the cross-frequency coupling analysis unit comprises an Intrinsic Mode Function selection module and the correlation coefficient matrix generation module;

the Intrinsic Mode Function selection module is configured to receive the plurality of different IMFs, and select n different IMFs from a same signal source or different signal sources;

the correlation coefficient matrix generation module is configured to combine the selected n different IMFs in pairs, quantify coupling relationship between the phase of low-frequency IMF and the amplitude of high-frequency IMF in each combination, and arrange the quantified coupling relationship to generate C(n,2) mxm correlation coefficient matrixes;

wherein C(n,2) is obtained by permutation and combination, that is, C(n,2)=n*(n−1)/2, the value of C(n,2) represents the number of the correlation coefficient matrixes; n is the number of the selected different IMFs; m is the number of the signal sources; mxm indicates that the number of rows and number of columns of the correlation coefficient matrixes is m; each element in the correlation coefficient matrix is defined as the difference between the probability density distribution of the phase of the j^{th} IMF and the amplitude of the i^{th} IMF (for short, the probability density distribution of the low-frequency phase and the high-frequency amplitude) and the uniform probability distribution (that is, the probability density distribution of Uniform Distribution), and is used as a measurement indicator for the coupling relationship between the phase of a low-frequency IMF and the amplitude of a high-frequency IMF from a same signal source or different signal sources, where 1≤i≤n−1, i<j≤n, and j is an integer greater than 1.

A functional network analysis method for complex network using the above functional network analysis system for complex network is further provided in the present application, comprising following steps:

S1: a multi-signal-source measurement unit measures signal data from signal sources in a complex network and sends the measured signal data of the signal sources to a signal decomposition unit;

S2: the signal decomposition unit receives the measured signal data, decomposes a plurality of different Mode Functions from the measured signal data by signal decomposition algorithms, and sends the plurality of different Mode Functions to a cross-frequency coupling analysis unit; and

S3: the cross-frequency coupling analysis unit receives the plurality of different Mode Functions, selects a certain number of different Mode Functions from a same signal source or different signal sources, combines the selected different Mode Functions in pairs, quantifies the coupling relationship between the phase of low-frequency Mode Function and the amplitude of high-frequency Mode Function in each combination, and arranges the quantified coupling relationship to generate a correlation coefficient matrix.

Optionally, the functional network analysis method for complex network further comprising:

S4: a functional network analysis unit receives the correlation coefficient matrix sent by the cross-frequency coupling analysis unit; obtain functional network attribute indicators based on Graph theory by using the correlation coefficient matrix as a calculation basis; and presents, in the form of graph, the functional network connection relationship described by the functional network attribute indicators.

Optionally, the functional network analysis method for complex network further comprising:

S5: a man-machine interaction device displays a man-machine interaction interface to a user; receives a graph, which presents the functional network connection relationship described by the functional network attribute indicators, sent by the functional network analysis unit; and displays the graph on the man-machine interaction interface.

Optionally, the functional network analysis method for complex network further comprising:

S0: the man-machine interaction device displays a man-machine interaction interface to a user; acquires a signal source measurement instructions input by the user on the man-machine interaction interface, and sends the signal source measurement instructions to the multi-signal-source measurement unit, wherein the signal source measurement instructions comprise the number of signal sources and the kind of signal sources.

Optionally, the S3 specifically comprising:

S31: a Mode Function selection module receives the plurality of different Mode Functions, and selects n different Mode Functions from a same signal source or different signal sources; and

S32: a correlation coefficient matrix generation module combines the selected n different Mode Functions in pairs, quantifies coupling relationship between the phase of low-frequency Mode Function and the amplitude of high-frequency Mode Function in each combination, and arrange the quantified coupling relationship to generate C(n,2) m×m correlation coefficient matrixes,

wherein, C(n,2) is obtained by permutation and combination, that is, C(n,2)=n*(n−1)/2, the value of C(n,2) represents the number of the correlation coefficient matrixes; n is the number of the selected different Mode Functions; m is the number of the signal sources; mxm indicates that the number of rows and number of columns of the correlation coefficient matrixes is m; each element in the correlation coefficient matrix is defined as the difference between the probability density distribution of the phase of the j^{th} Mode Function and the amplitude of the i^{th} Mode Function (for short, the probability density distribution of the low-frequency phase and the high-frequency amplitude) and the uniform probability distribution (that is, the probability density distribution of Uniform Distribution), and is used as a measurement indicator for the coupling relationship between the phase of a low-frequency Mode Function and the amplitude of a high-frequency Mode Function from a same signal source or different signal sources, where 1≤i≤n−1, i<j≤n and j is an integer greater than 1.

### BRIEF DESCRIPTION OF THE DRAWINGS

FIG. 1 is a schematic structure diagram of a functional network analysis system for complex network in one implementation;

FIG. 2 is a flowchart of afunctional network analysis system for complex network in one implementation;

FIG. 3 shows a 21-electrode EEG of an elderly healthy subject, with his eyes closed, in Embodiment 1;

FIG. 4 shows IMFs obtained from the EEG measured by the FP1 electrode of FIG. 3 by EMD;

FIG. 5a shows the probability density distribution of the low-frequency phase and the high-frequency amplitude of six cross-band combinations of the first through sixth IMFs of the IMFs of FIG. 4;

FIG. 5b shows the probability density distribution of the low-frequency phase and the high-frequency amplitude of nine cross-band combinations of the first through sixth IMFs of the IMFs of FIG. 4;

FIG. 6a shows correlation coefficient matrixes of six cross-band combinations of the first through sixth IMFs from 21 signal sources (i.e., 21 electrodes) in Embodiment 1;

FIG. 6b shows correlation coefficient matrixes of nine cross-band combinations of the first through sixth IMFs from 21 signal sources (i.e., 21 electrodes) in Embodiment 1;

FIG. 7a is topography of strength of node, one of the functional network attribute indicators for the functional network connection relationship of six cross-band combinations in the brain , in Embodiment 1; and

FIG. 7b is topography of strength of node, one of the functional network attribute indicators for the functional network connection relationship of nine cross-band combinations in the brain, in Embodiment 1, in which:

1: multi-signal-source measurement unit; 2: a signal decomposition unit; 3: a cross-frequency coupling analysis unit; 4: functional network analysis unit; 5: man-machine interaction device.

### DETAILED DESCRIPTION OF THE PRESENT INVENTION

In the following, the present application is described in detail by exemplary implementation, however, it should be understood that, the element, structure and features of one implementation may be beneficially incorporated into the other implementations without further recitation.

In the depict of the present application, it should be noted that terms “system”, “unit”, “module” used herein are one method to distinguish different components, elements, parts, section or assembly of different level in ascending. However, the terms may be displaced by other expression if they may achieve the same purpose. It will be understood that when a unit, module or block is referred to as being “on”, “connected to”, or “coupled to” another unit, module, it may be directly on, connected to or coupled to, or communicate with the other unit, module, or block, or an intervening unit, module, or block may be present, unless the context clearly indicates otherwise.

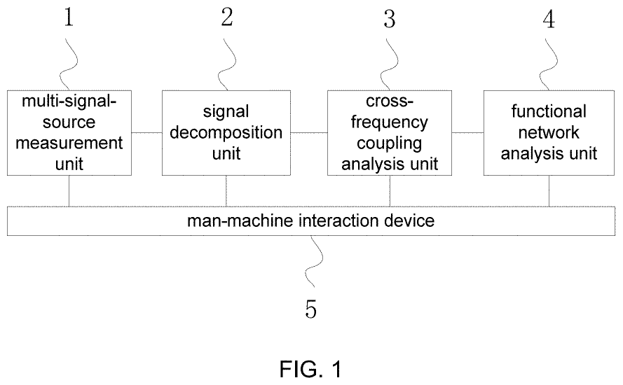

In one aspect, the present application provides a functional network analysis system for complex network. As shown in FIG. 1, the functional network analysis system for complex networkcomprisesa multi-signal-source measurement unit 1, a signal decomposition unit 2 and a cross-frequency coupling analysis unit 3, wherein the multi-signal-source measurement unit 1, the signal decomposition unit 2 and the cross-frequency coupling analysis unit 3 are connected successively;

the multi-signal-source measurement unit 1 is configured to measure signal data from signal sources in a complex network and send the measured signal dataof the signal sources to the signal decomposition unit 2;

the signal decomposition unit 2 is configured to receive the measured signal data, decompose a plurality of different Mode Functions from the measured signal data by signal decomposition algorithms, and send the plurality of different Mode Functions to the cross-frequency coupling analysis unit 3; and

the cross-frequency coupling analysis unit 3 is configured to receive the plurality of different Mode Functions, select a certain number of different Mode Functions from a same signal source or different signal sources, combine the selected different Mode Functions in pairs, quantify the coupling relationship between the phase of low-frequency Mode Function and the amplitude of high-frequency Mode Function in each combination, and arrange the quantified coupling relationship to generate a correlation coefficient matrix.

The multi-signal-source measurement unit 1 comprises an array of sensing elements that are configured to measure signal sources of a same kind from different locations or different elements or signal sources of different kinds from a same location or a same element in a complex network. The signal decomposition unit 2 may be a combination of micro-processing elements and a plurality of software and have a function of signal decomposition. By the functional network analysis system for complex network in this implementation, the coupling relationship between signal sources of a same kind at different locations can be analyzed, and the coupling relationship between signal sources of different kinds at a same location or at different locations can also be analyzed, wherein the coupling relationship between multiple signal sources is defined as cross-frequency phase-amplitude coupling of cross signal sourcesin present application, which can reflect the dynamic properties of functional network links between different frequency bands in a complex system. The description of brain network may be divided into three types: structural network, functional network and effective network, wherein the functional network, as an undirected network, is used to describe the statistical connection relationship between nodes in the cortical neural network. In the real world, the complex networks which can be described by functional networks, such as, brain network, regional oceanography network, mechanical structures network, internet network and sound field network, also can be analyzed by the functional network analysis system provided in this implementation. Mode Functions which are extracted from signal data is indicative of oscillatory modes in the signal.

In some implementations, the cross-frequency coupling analysis unit 3 comprises a Mode Function selection module and a correlation coefficient matrix generation module. The Mode Function selection module is configured to receive the plurality of different Mode Functions, and select n different Mode Functions from a same signal source or different signal sources. The correlation coefficient matrix generation module is configured to combine the selected n different Mode Functions in pairs, quantify coupling relationship between the phase of low-frequency Mode Function and the amplitude of high-frequency Mode Function in each combination, and arrange the quantified coupling relationship to generate C(n,2) m×m correlation coefficient matrixes, wherein C(n,2) is obtained by permutation and combination, that is, C(n,2)=n*(n-1)/2, the value of C(n,2) represents the number of the correlation coefficient matrixes; n is the number of the selected different Mode Functions; m is the number of the signal sources; mxm indicates that the number of rows and number of columns of the correlation coefficient matrixes is m; each element in the correlation coefficient matrix is defined as the difference between the probability density distribution of the phase of the j^{th} Mode Function and the amplitude of the i^{th} Mode Function (for short, the probability density distribution of the low-frequency phase and the high-frequency amplitude) and the uniform probability distribution (that is, the probability density distribution of Uniform Distribution), and is used as a measurement indicator for the coupling relationship between the phase of a low-frequency Mode Function and the amplitude of a high-frequency Mode Function from a same signal source or different signal sources, where 1≤i≤n−1, i<j≤n, and j is an integer greater than 1. The higher the order of a Mode Function is, the lower the frequency is. The probability density distribution of the low-frequency phase and the high-frequency amplitude may use a single statistical correlation coefficient to represent its coupling properties, such as the KL distance (Kullback-Leibler distance) representing relative entropy. The KL distance represents the difference between the probability density distribution of the low-frequency phase and the high-frequency amplitude and that of the uniform probability distribution. The greater the difference, that is, the greater the KL distance, it means that the probability density of the high-frequency Mode Function amplitude is concentrated in a specific phase which indicating that there is coupling relationship between the phase of the low-frequency Mode Function and the amplitude of the high-frequency Mode Function. Conversely, if the probability density distribution of the low-frequency phase and the high-frequency amplitude approximates the uniform probability distribution, that is, the KL distance is less, it is indicated that there is a weak coupling relationship between the phase of the low-frequency Mode Function and the amplitude of the high-frequency Mode Function. Particularly, the coupling relationship between the phase of the low-frequency Mode Function and the amplitude of the high-frequency Mode Function at a specific frequency band can be presented in the form of two-dimensional holographic spectrum. The quantitative indicators of the cross-frequency phase-amplitude coupling analysis in the two-dimensional holographic spectrum include cross-frequency coupling relationship in two dimensions. The first dimension is the order of the Mode Function for the low-frequency phase, and the second dimension is the order of the Mode Function for the high-frequency amplitude. The Mode Function selection module may be a window with frequency distribution display function for different Mode Functions and Mode Function selection function to select the Mode Functions, so as to select Mode Function according to the analysis needs. The correlation coefficient matrix generation module may be a micro-processing element with a distribute calculating function, which can simultaneously and quickly calculate the functional coupling relationship between multiple signal sources, and arrange the coupling relationship in a preset layout to generate thecorrelation coefficient matrix.

Specifically, the signal decomposition algorithm used by the signal decomposition unit may be a non-adaptive digital filtering technique based on linear disassembly methods such as Fourier transform or Wavelets theory, or may be a non-linear data adaptive decomposition technique, for example, an Empirical Mode Decomposition algorithm (EMD) with adaptive functions. For the non-adaptive digital filtering technique, by setting different filtering frequency bands for the signal decomposition unit, the Mode Functions at different bands are decomposed.

In some implementations, the signal decomposition algorithm used by the signal decomposition unitmay be EMD. That is, the signal decomposition unit 2 decomposes a plurality of different Intrinsic Mode Functions (IMFs) from the signal data of the signal sources by EMD, and sends the plurality of different IMFs to the cross-frequency coupling analysis unit; and

the cross-frequency coupling analysis unit 3 is configured to receive the plurality of different IMFs, select a certain number of IMFs from a same signal source or different signal sources, combine the selected different IMFs in pairs, quantify coupling relationship between the phase of low-frequency IMF and the amplitude of high-frequency IMF in each combination, and arrange the quantified coupling relationship to generate a correlation coefficient matrix.

EMD belongs to one of the non-linear data adaptive decomposition techniques. The non-linear data adaptive decomposition technique can meet the four necessary conditions for non-linear non-stationary data decomposition: completeness, orthogonality, locality and adaptability. It is more suitable for the decomposition of non-linear and non-stationary data in complex networks. In some implementations, the functional network analysis system for complex network uses the non-linear data adaptive decomposition technique to decompose the signal data in signal sources. The coupling relationship between the frequency bands can be analyzed systematically and automatically. The functional connection relationship between signal sources is represented by the correlation coefficient matrix. The so-called “automatically” in the present application refers to the disassembly of components (Mode Function) at each frequency band is based on the characteristics of the data itself, instead of using a liner band-pass filtering bank and manually setting the frequency band in advance for the linear band-pass filtering bank to perform data decomposition.

As further optimization of the above implementations, the cross-frequency coupling analysis unit 3 comprises an Intrinsic Mode Function selection module and the correlation coefficient matrix generation module. The Intrinsic Mode Function selection module is configured to receive the plurality of different IMFs, and select n different IMFs from a same signal source or different signal sources. The correlation coefficient matrix generation module is configured to combine the selected n different IMFs in pairs, quantify coupling relationship between the phase of low-frequency IMF and the amplitude of high-frequency IMF in each combination, and arrange the quantified coupling relationship to generate C(n,2) m×m correlation coefficient matrixes.

The number of IMFs decomposable from data in each signal source is determined according to the number of data in the signal source. For example, N is the number of data in one signal, given that N is 1024 and the initial frequency of the signal source is H, then, since one oscillation period includes at least four data, the data in this signal source can be decomposed into eight IMFs at most. That is, eight different IMFs can be obtained, and the frequency of each IMF gradually decreases from the first to the eighth. The first IMF (that is the first Intrinsic Mode Function) has a total of 256 oscillation periods and the frequency is H/4; the second IMF (that is the second Intrinsic Mode Function) has a total of 128 oscillation periods and the frequency is H/8; the third IMF (that is the third Intrinsic Mode Function) has a total of 64 oscillation periods and the frequency is H/16; and so forth. The oscillation period and frequency of each of IMFs can be obtained. If the number of data in a signal source is 1024≤N<2048, the data in this signal source can be decomposed into eight IMFs at most. The multi-signal-source measurement unit is selected according to the application field. For example, when it is applied to the functional network analysis for the brain, a brainwave apparatus is selected as the multi-signal-source measurement unit to obtain EEG data from different locations of the brain.

In some implementations, the functional network analysis system for complex network further comprises a functional network analysis unit 4 which is connected to the cross-frequency coupling analysis unit 3.

The functional network analysis unit 4 is configured to receive the correlation coefficient matrix sent by the cross-frequency coupling analysis unit 3; obtain functional network attribute indicators based on Graph theory by using the correlation coefficient matrix as a calculation basis; and present, in the form of graph, the functional network connection relationship described by the functional network attribute indicators. Graph theory is an important tool for describing network characteristics. It takes graphs as research objects. Graphs in Graph theory are composed by a number of given nodes and edges connecting two nodes. Such graphs are usually used to describe a specific relationship between some objections. The nodes represent objections, and the edges connecting the two nodes are used to indicate that there exists the specific relationship between the corresponding two objections. The functional network attribute indicators are commonly used measurement indicators in complex network analysis based on Graph theory. The functional network attribute indicators can be calculated from the correlation coefficient matrix. The functional network attribute indicators mainly comprise clustering coefficient, characteristic path length, and degree of node. The clustering coefficient is a coefficient indicating the degree of aggregation of nodes in a graph, and is used to measure the degree of aggregation of a complex network. In the network, any two nodes are selected, and the minimum number of edges connecting the two nodes is defined as the path length of the two nodes. The average path length of all node pairs in the network is defined as the characteristic path length of the network, which is used to characterize the global characteristics of the network. The degree of node includes degree centrality of node and strength of node. The degree centrality of node refers to the number of edges associated with the node, also known as the degree of linkage. The strength of node refers to the sum of weights for edges connected to the node in a weighted complex network. The functional network analysis unit combines calculation elements, calculation methods, data structure protocols, and 3D drawing functions to transform the correlation coefficient matrix into attribute indicators of node and edge, reflecting the functional connection relationship of complex networks. In some implementations, the functional network analysis system for complex network further comprises a man-machine interaction device 5 connected to the functional network analysis unit 4, the man-machine interaction device 5 is configured to display a man-machine interaction interface to a user; receive a graph, which presents the functional network connection relationship described by functional network attribute indicators, sent by the functional network analysis unit 4; and display the graph on the man-machine interaction interface.

In some implementations, the man-machine interaction device 5 is connected to the multi-signal-source measurement unit 1, and is configured to acquire signal source measurement instructions input by the user on the man-machine interaction interface and send the signal source measurement instructions to the multi-signal-source measurement unit 1, wherein the signal source measurement instructions comprise the number of signal sources and the kind of signal sources.

The man-machine interaction device 5 may also be connected to the signal decomposition unit 2 and the cross-frequency coupling analysis unit 3. It may allow the user to set setting parameters and output options for the units. For example, the order and the number of Mode Functions selected by the cross-frequency coupling analysis unit 3 can be set. The man-machine interaction device 5 may be a man-machine interface containing drawing software, data structure protocols, data output devices, displays, parameter input windows, function setting menus and hardware operating interfaces (for example, keyboard, mouse, rocker or trackball).

In the present application, by setting the man-machine interaction device, a friendly graphical man-machine interaction interface is provided, by which the user sets the number of signal sources, the kind of signal sources, setting parameters and output options for the units.

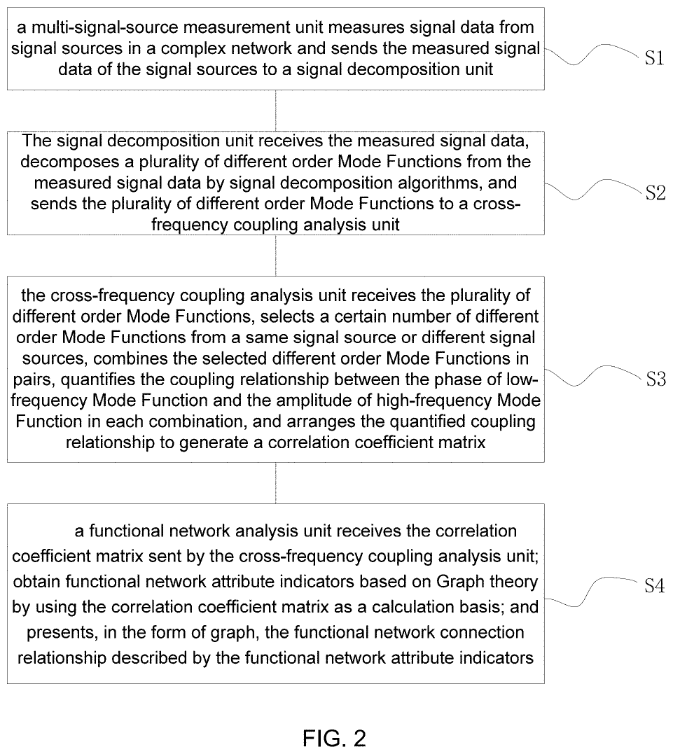

In another aspect, the present application further provides a functional network analysis method for a complex network, which uses the functional network analysis system for complex network described above. As shown in FIG. 2, the method successively comprises the following steps:

S1: amulti-signal-source measurement unit 1 measures signal data from signal sources in a complex network and sends the measured signal data of the signal sources to a signal decomposition unit 2;

S2: the signal decomposition unit 2 receives the measured signal data, decomposes a plurality of different Mode Functions from the measured signal data by signal decomposition algorithms, and sends the plurality of different Mode Functions to across-frequency coupling analysis unit 3; and

S3: the cross-frequency coupling analysis unit 3 receives the plurality of different Mode Functions, selects a certain number of different Mode Functions from a same signal source or different signal sources, combines the selected different Mode Functions in pairs, quantifies the coupling relationship between the phase of low-frequency Mode Function and the amplitude of high-frequency Mode Function in each combination, and arranges the quantified coupling relationship to generate a correlation coefficient matrix.

Optionally, the method further comprises SO: a man-machine interaction device 5 displays a man-machine interaction interface to a user; acquires a signal source measurement instructions input by the user on the man-machine interaction interface, and sends the signal source measurement instructions to the multi-signal-source measurement unit 1, wherein the signal source measurement instructions comprise the number of signal sources and the kind of signal sources.

Optionally, the S3 specifically comprises following steps:

S31: a Mode Function selection module receives the plurality of different Mode Functions, and selects n different Mode Functions from a same signal source or different signal sources; and

S32: a correlation coefficient matrix generation module combines the selected n different Mode Functions in pairs, quantifies coupling relationship between the phase of low-frequency Mode Function and the amplitude of high-frequency Mode Function in each combination, and arrange the quantified coupling relationship to generate C(n,2) m×m correlation coefficient matrixes,

wherein, C(n,2) is obtained by permutation and combination, that is, C(n,2)=n*(n−1)/2, the value of C(n,2) represents the number of the correlation coefficient matrixes; n is the number of the selected different Mode Functions; m is the number of the signal sources; mxm indicates that the number of rows and number of columns of the correlation coefficient matrixes is m; each element in the correlation coefficient matrix is defined as the difference between the probability density distribution of the phase of the j^{th} Mode Function and the amplitude of the i^{th} Mode Function (for short, the probability density distribution of the low-frequency phase and the high-frequency amplitude) and the uniform probability distribution (that is, the probability density distribution of Uniform Distribution), and is used as a measurement indicator for the coupling relationship between the phase of a low-frequency Mode Function and the amplitude of a high-frequency Mode Function from a same signal source or different signal sources, where 1≤i≤n−1, i<j≤n, and j is an integer greater than 1.

Optionally, the method further comprises S4: a functional network analysis unit 4 receives the correlation coefficient matrix sent by the cross-frequency coupling analysis unit 3; obtain functional network attribute indicators based on Graph theory by using the correlation coefficient matrix as a calculation basis; and presents, in the form of graph, the functional network connection relationship described by the functional network attribute indicators.

Further, the method further comprises S5: the man-machine interaction device 5 displays a man-machine interaction interface to a user; receives a graph, which presents the functional network connection relationship described by functional network attribute indicators, sent by the functional network analysis unit 4; and displays the graph on the man-machine interaction interface.

### Embodiment 1

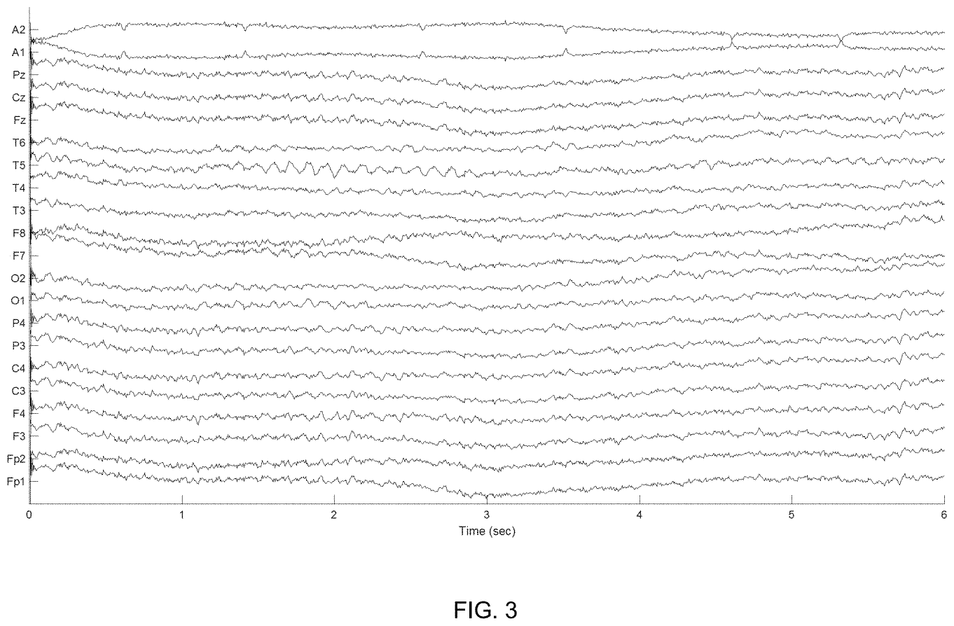

In this embodiment, the analysis on a 21-electrode EEG data of patients with Alzheimer's disease is used as an application example. The subjects were divided into five groups according to age and degree of brain degradation in the database: young healthy control group, elderly healthy control group, patients with mild mental deterioration, patients with moderate Alzheimer's disease, and patients with severe Alzheimer's disease. 21-electrode EEGs were obtained by a brainwave apparatus (i.e., a multi-signal-source measurement unit) with the eyes of the subjects closed. The sampling frequency was 200 Hz. FIG. 3 shows EEG measurement data of an elderly healthy control group, with the eyes closed. The sampling frequency is 200 Hz and the sampling time is 6 s. Therefore, the amount of data in each signal source (that is each electrode) is 1200. The letter of the vertical scale in FIG. 3 represents the location of the electrode when obtaining the EEG. Specifically, A2 represents the right earlobe (reference electrode), A1 represents the left earlobe (reference electrode), Pz represents the parietal midline, Cz represents the central midline, Fz represents the frontal midline, T6 represents the right posterior temporal, T5 represents the left posterior temporal, T4 represents the right mid-temporal, T3 represents the left mid-temporal, F8 represents the right anterior temporal, F7 represents the left anterior temporal, O2 represents the right occipital, 01 represents the left occipital, P4 represents the right parietal, P3 represents the left parietal, C4 represents the right central, C3 represents the left central, F4 represents the right frontal, F3 represents the left frontal, Fp2 represents the right frontal pole, and Fp1 represents the left frontal pole.

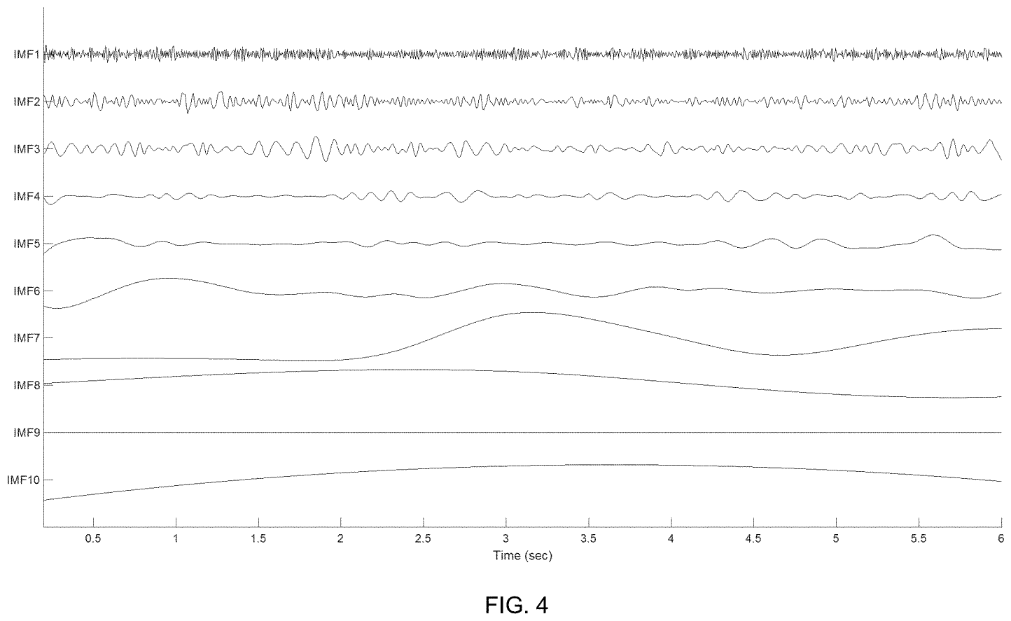

The EEG data (i.e., the measured signal data of the signal sources) measured by each electrode is sent to a signal decomposition unit and the signal decomposition unit decomposes the measured signal data of each signal source by signal decomposition algorithms. In this embodiment, the signal decomposition unit decomposes a plurality of different IMFs from the measured signal data by the EMD. As shown in FIG. 4, eight different IMFs are decomposed from the EEG data measured by the FP1 electrode in FIG. 3 by the signal decomposition unit. As shown in FIG. 4, the vertical coordinate IMF1 represents the first IMF. Similarly, IMF2, IMF3, IMF4, IMF5, IMF6, IMF7, IMF8, respectively, represent the second to eighth IMFs. It should be noted that, since IMF9 and IMF10 in the drawing do not have a complete oscillation period, they cannot be regarded as the ninth and tenth IMFs. Of course, by the decomposition of the signal decomposition function unit, eight IMFs can be extracted from the EEG data measured by each electrode of the 21 electrodes.

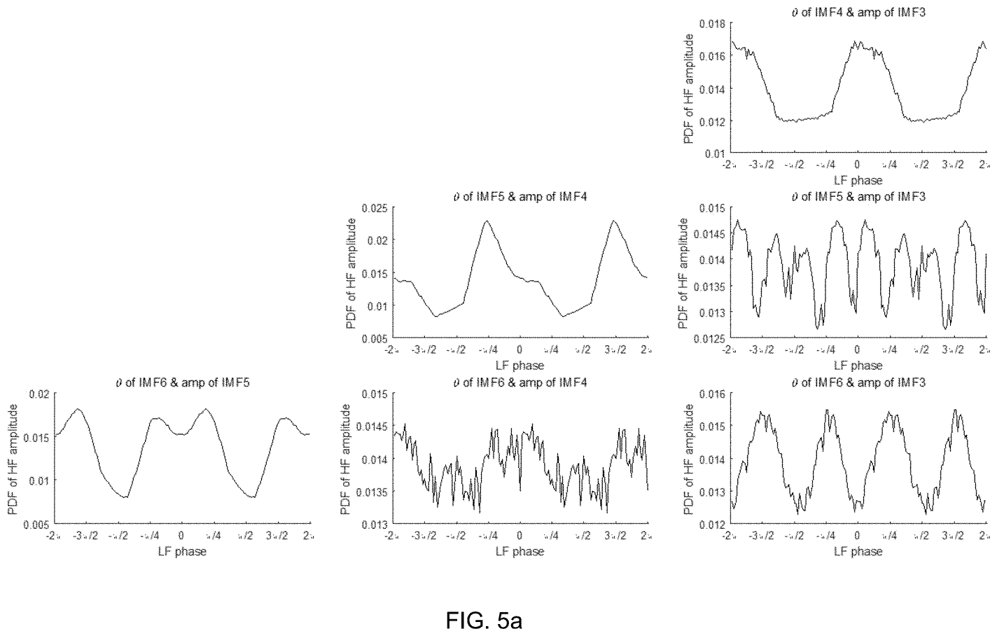

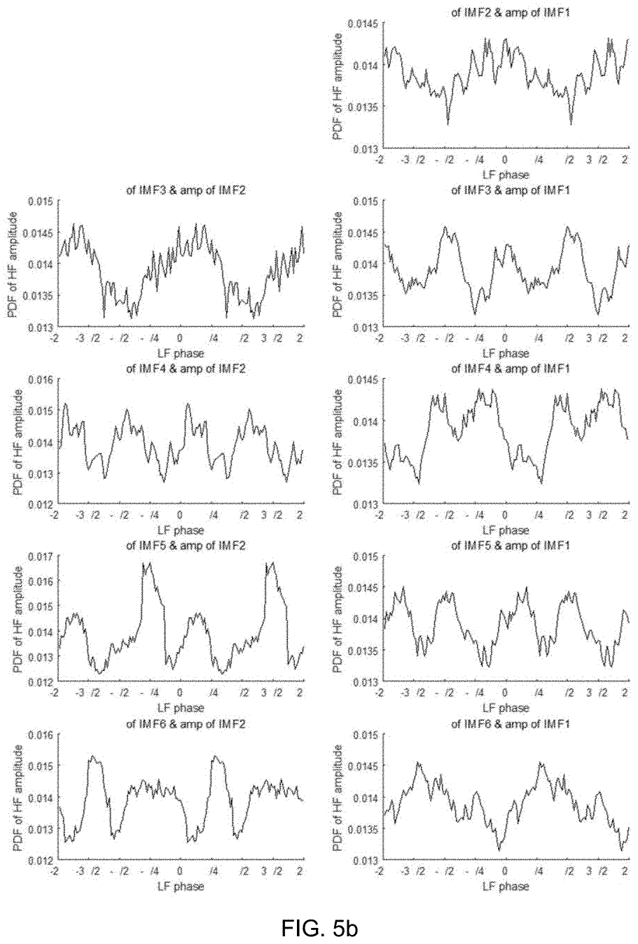

The signal decomposition unit sends the decomposition result to the cross-frequency coupling analysis unit. The Intrinsic Mode Function selection module of the cross-frequency coupling analysis unit can select n different IMFs from a plurality of different IMFs from a same signal source or different signal sources. In this embodiment, the Intrinsic Mode Function selection module selects the first through sixth IMFs from eight different IMFs from a single signal source FP1; and then the correlation coefficient matrix generation module automatically obtains, by permutation and combination, fifteen combinations of different IMFs (i.e., cross-band combinations), and then quantifies the coupling relationship between the phase of the low-frequency Mode Function and the amplitude of the high-frequency Mode Function in the fifteen combinations. In this embodiment, the coupling relationship between the phase of the low-frequency Mode Function and the amplitude of the high-frequency Mode Function (hereinafter referred to as the coupling relationship between the low-frequency phase and the high-frequency amplitude) is quantified by the KL distance. That is, by calculating the difference between the probability density distribution of the low-frequency phase and the high-frequency amplitude and the uniform probability distribution, the coupling relationship between the low-frequency phase and the high-frequency amplitude is measured. As shown in FIGS. 5a and 5b, the probability density distribution of the low-frequency phase and the high-frequency amplitude of fifteen cross-band combinations of the first through sixth IMFs from a single signal source are shown. Of course, the Intrinsic Mode Function selection module can also select six different IMFs from different signal sources. For example, the first, second and third IMFs are selected from the eight different IMFs decomposed from the measured signal data of the signal source measured by the FP1 electrode; and the fourth, fifth and sixth IMFs are selected from the eight different IMFs decomposed from the measured signal data of the signal source measured by the FP2 electrode. Then, the correlation coefficient matrix generation module combines these six different IMFs in pairs to obtain fifteen cross-band combinations. Then, the coupling relationship between the low-frequency phase and high-frequency amplitude in these fifteen cross-band combinations is quantified.

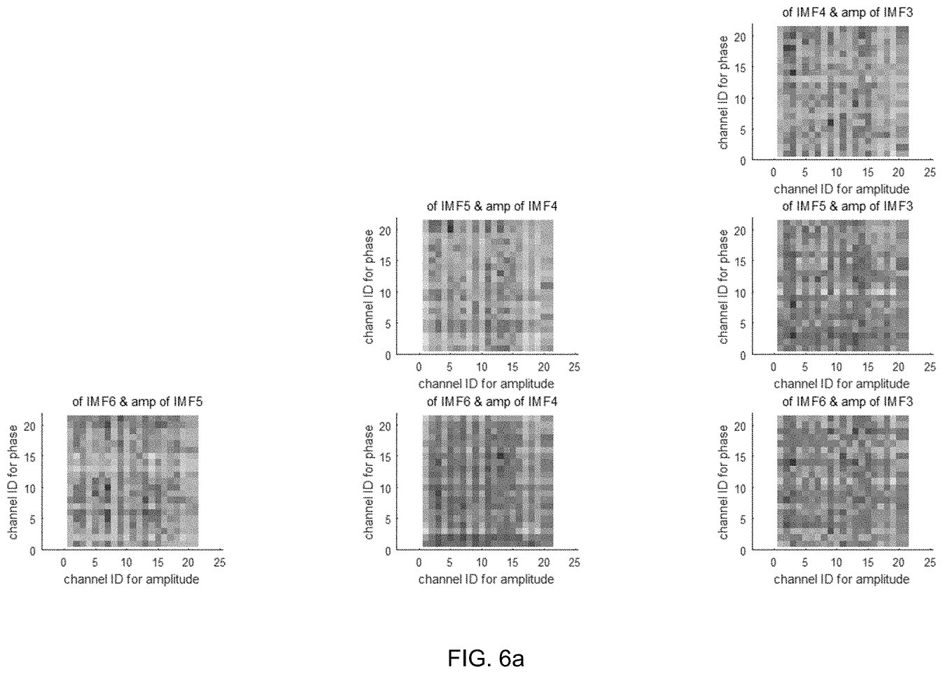

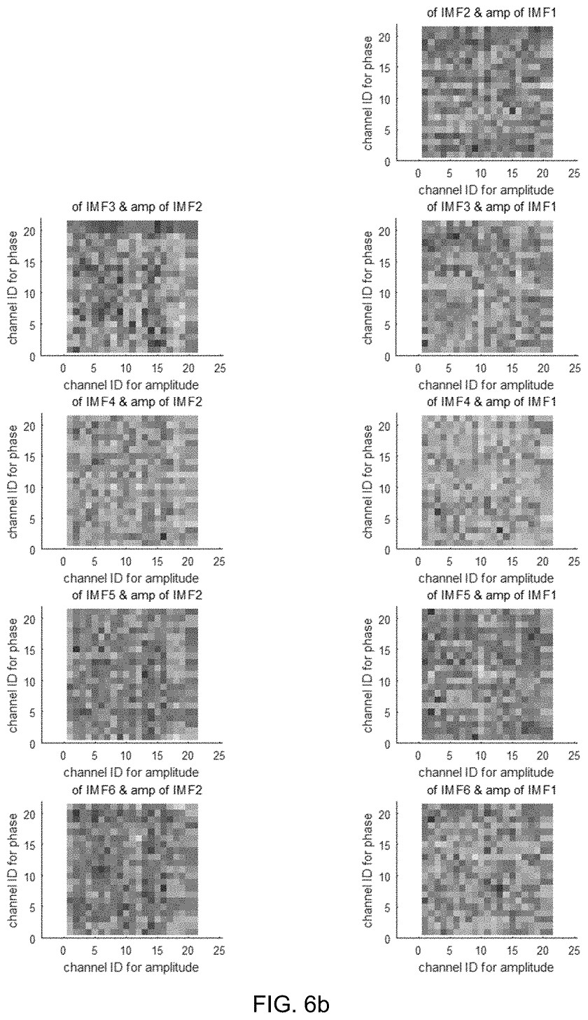

The cross-frequency coupling analysis unit arbitrarily selects six different IMFs from the IMFs decomposed from the measured signal data of the signal sources measured by the 21 electrodes. There are total 21*21 selection methods (the six different IMFs may be from a same signal source or from different signal sources). Each selection method can obtain fifteen cross-band combinations. Then, the coupling relationship between the low-frequency phase and high-frequency amplitude in the fifteen cross-band combinations corresponding to each selection method is quantified. The KL distance is used as a measurement indicator for the coupling relationship. A total of 21*21 KL distance values can be obtained. By arranging the KL distance of the eachcross-band combinations from the 21 signal sources, the matrix of coupling relationship can be obtained, which is called a correlation coefficient matrix, as shown in FIGS. 6a and 6b. FIGS. 6a and 6b show the correlation coefficient matrix of 15 cross-band combinations of the first through sixth IMFs from 21 signal sources (i.e., 21 EEG electrodes). Each correlation coefficient matrix is a 21*21 matrix. In the drawing, the color depth is used to indicate the size of the coupling relationship of the cross-band combinations. The deeper the color is, the greater the coupling relationship is.

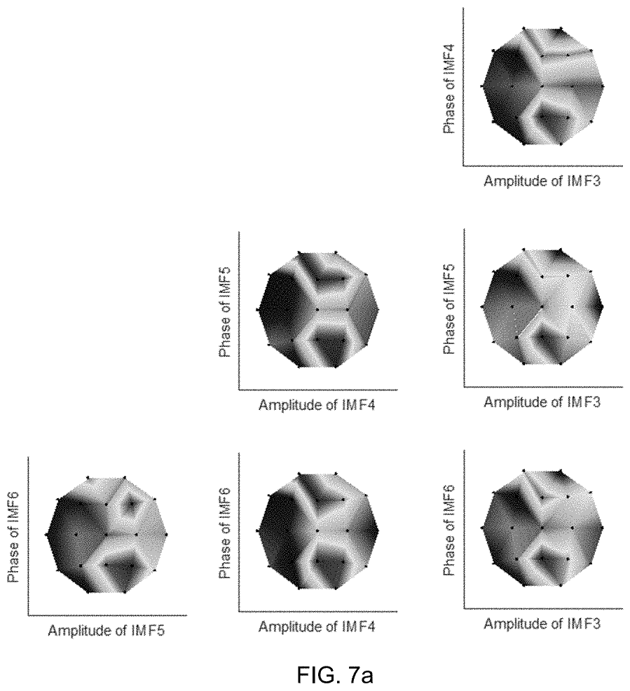

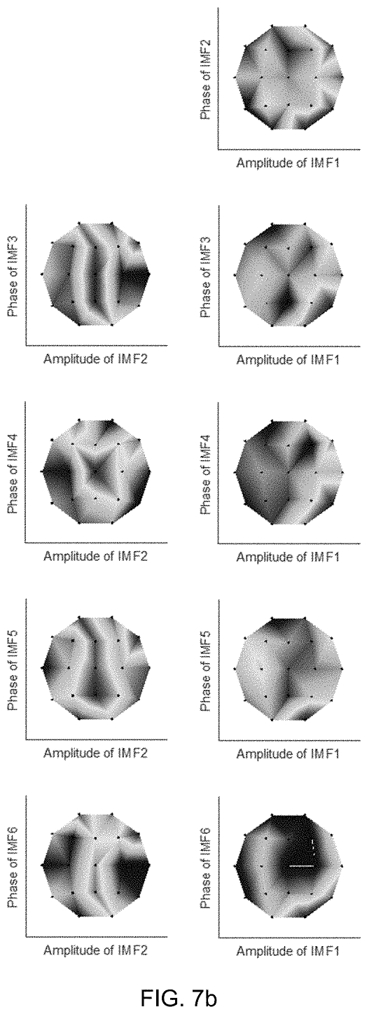

The correlation coefficient matrix can represent the functional network connection relationship between different signal sources. Through the application of Graph Theory, complex functional network connection relationships can be simplified. When it is applied to the functional analysis of the complex network in the brain, each brain region in the human brain or a small division of the brain is considered as a node, the connection between the brain regions is considered as edges, thus build a functional network of the brain network. In this embodiment, the cross-frequency coupling analysis unit sends the generated 21*21 correlation coefficient matrixes to the functional network analysis unit. Based on the 21*21 correlation coefficient matrixes, the functional network analysis unit obtains functional network attribute indicators which are based on Graph theory by using the brain regions corresponding to the 21 electrodes as nodes, to quantify the functional network connection relationship in the brain. These quantitative indicators may comprise characteristic path length, clustering coefficient, characteristic path length, and strength of node. The characteristic path length and clustering coefficient can represent the overall characteristics of the system; the strength of node can highlight the characteristics of a single electrode. FIGS. 7a and 7b is topography of strength of node of each electrode. In FIGS. 7a and 7b, the strength of node reflects the relative functional connection strength between the brain regions corresponding to each electrode under the set test conditions. Each picture in FIGS. 7a and 7b indicates that, at a specific frequency band, brain regions corresponding to different electrodes have different functional connection strengths. The complex brain functional connection characteristics can be intuitively obtained from topography. Topography of other functional network attribute indicators can also be obtained. The principle is the same as the strength of node topography, and will not be repeated here.

The functional network analysis system and analysis method provided in the present application can also be applied in other fields, such as network analysis of regional oceans. By using values of temperature, salinity, and physical quantities such as wave height, velocity and sound waves at different regional points as data in signal sources, the coupling relationship between same or different physical quantities at different time scales is discussed, in order to obtain the network characteristics of regional oceans.

The functional network analysis system and analysis method provided in the present application can also be applied to the network analysis of mechanical structures. By using values of physical quantities such as temperature, vibration, stress and sound as data in signal sources, the coupling between the physical quantities at different time scales is discussed, in order to obtain the network characteristics of mechanical structures.

The above embodiments are only used to illustrate the technical solutions of the present application, and not intended to limit the present application. The present application uses the empirical mode decomposition algorithm as a representative of adaptive non-linear data decomposition methods. However, it does not exclude the use of extensions or improvements to the EMD or different signal decomposition algorithms as the basis of the data processing in the present application. In particular, although the present application has been described in detail with reference to the foregoing embodiments, it should be understood by a person of ordinary skill in the art that the technical solutions described in the foregoing embodiments can be modified, or some of the technical features thereof can be equivalently replaced. These modifications or replacements will not make the essence of the corresponding technical solutions depart from the spirit and scope of the technical solutions of the embodiments of the present application.

1. A functional network analysis system for complex network, comprising a multi-signal-source measurement unit, a signal decomposition unit and a cross-frequency coupling analysis unit, wherein the multi-signal-source measurement unit, the signal decomposition unit and the cross-frequency coupling analysis unit are connected successively;
the multi-signal-source measurement unit is configured to measure signal data from signal sources in a complex network and send the measured signal data of the signal sources to the signal decomposition unit;
the signal decomposition unit is configured to receive the measured signal data, decompose a plurality of different Mode Functions from the measured signal data by signal decomposition algorithms, and send the plurality of different Mode Functions to the cross-frequency coupling analysis unit; and
the cross-frequency coupling analysis unit is configured to receive the plurality of different Mode Functions, select a certain number of different Mode Functions from a same signal source or different signal sources, combine the selected different Mode Functions in pairs, quantify the coupling relationship between the phase of low-frequency Mode Function and the amplitude of high-frequency Mode Function in each combination, and arrange the quantified coupling relationship to generate a correlation coefficient matrix.

2. The functional network analysis system for complex network of claim 1 comprising a functional network analysis unit which is connected to the cross-frequency coupling analysis unit;
wherein the functional network analysis unit is configured to receive the correlation coefficient matrix sent by the cross-frequency coupling analysis unit; obtain functional network attribute indicators based on Graph theory by using the correlation coefficient matrix as a calculation basis; and present, in the form of graph, the functional network connection relationship described by the functional network attribute indicators.

3. The functional network analysis system for complex network of claim 2 comprising a man-machine interaction device connected to the functional network analysis unit, wherein the man-machine interaction device is configured to display a man-machine interaction interface to a user, receive a graph, which presents the functional network connection relationship described by functional network attribute indicators, sent by the functional network analysis unit, and display the graph on the man-machine interaction interface.

4. The functional network analysis system for complex network of claim 3, wherein the man-machine interaction device is connected to the multi-signal-source measurement unit, and is configured to acquire signal source measurement instructions input by the user on the man-machine interaction interface and send the signal source measurement instructions to the multi-signal-source measurement unit, wherein the signal source measurement instructions comprise the number of signal sources and the kind of signal sources.

5. The functional network analysis system for complex network of claim 1, wherein the cross-frequency coupling analysis unit comprises a Mode Function selection module and a correlation coefficient matrix generation module;
the Mode Function selection module is configured to receive the plurality of different Mode Functions, and select n different Mode Functions from a same signal source or different signal sources;
the correlation coefficient matrix generation module is configured to combine the selected n different Mode Functions in pairs, quantify coupling relationship between the phase of low-frequency Mode Function and the amplitude of high-frequency Mode Function in each combination, and arrange the quantified coupling relationship to generate C(n,2) m×m correlation coefficient matrixes;
wherein C(n,2) is obtained by permutation and combination, that is, C(n,2)=n*(n−1)/2, the value of C(n,2) represents the number of the correlation coefficient matrixes; n is the number of the selected different Mode Functions; m is the number of the signal sources; mxm indicates that the number of rows and number of columns of the correlation coefficient matrixes is m; each element in the correlation coefficient matrix is defined as the difference between the probability density distribution of the phase of the j^{th} Mode Function and the amplitude of the i^{th} Mode Function (for short, the probability density distribution of the low-frequency phase and the high-frequency amplitude) and the uniform probability distribution (that is, the probability density distribution of Uniform Distribution), and is used as a measurement indicator for the coupling relationship between the phase of a low-frequency Mode Function and the amplitude of a high-frequency Mode Function from a same signal source or different signal sources, where 1≤i≤n−1, i<j≤n, and j is an integer greater than 1.

6. The functional network analysis system for complex network of claim 2, wherein the signal decomposition unit decomposes a plurality of different Intrinsic Mode Functions (IMFs) from the signal data of the signal sources by EMD, and sends the plurality of different IMFs to the cross-frequency coupling analysis unit; and
the cross-frequency coupling analysis unit is configured to receive the plurality of different IMFs, select a certain number of IMFs from a same signal source or different signal sources, combine the selected different IMFs in pairs, quantify coupling relationship between the phase of low-frequency IMF and the amplitude of high-frequency IMF in each combination, and arrange the quantified coupling relationship to generate a correlation coefficient matrix.

7. The functional network analysis system for complex network of claim 6, wherein the cross-frequency coupling analysis unit comprises an Intrinsic Mode Function selection module and the correlation coefficient matrix generation module;
the Intrinsic Mode Function selection module is configured to receive the plurality of different IMFs, and select n different IMFs from a same signal source or different signal sources;
the correlation coefficient matrix generation module is configured to combine the selected n different IMFs in pairs, quantify coupling relationship between the phase of low-frequency IMF and the amplitude of high-frequency IMF in each combination, and arrange the quantified coupling relationship to generate C(n,2) mxm correlation coefficient matrixes;
wherein, C(n,2) is obtained by permutation and combination, that is, C(n,2)=n*(n−1)/2, the value of C(n,2) represents the number of the correlation coefficient matrixes; n is the number of the selected different IMFs; m is the number of the signal sources; mxm indicates that the number of rows and number of columns of the correlation coefficient matrixes is m; each element in the correlation coefficient matrix is defined as the difference between the probability density distribution of the phase of the j^{th} IMF and the amplitude of the i^{th} IMF (for short, the probability density distribution of the low-frequency phase and the high-frequency amplitude) and the uniform probability distribution (that is, the probability density distribution of Uniform Distribution), and is used as a measurement indicator for the coupling relationship between the phase of a low-frequency IMF and the amplitude of a high-frequency IMF from a same signal source or different signal sources, where 1≤i≤n−1, i<j≤n, and j is an integer greater than 1.

8. A functional network analysis method for complex network using the functional network analysis system according to claim 1, comprising the following steps:
S1: a multi-signal-source measurement unit measures signal data from signal sources in a complex network and sends the measured signal data of the signal sources to a signal decomposition unit;
S2: the signal decomposition unit receives the measured signal data, decomposes a plurality of different Mode Functions from the measured signal data by signal decomposition algorithms, and sends the plurality of different Mode Functions to a cross-frequency coupling analysis unit; and
S3: the cross-frequency coupling analysis unit receives the plurality of different Mode Functions, selects a certain number of different Mode Functions from a same signal source or different signal sources, combines the selected different Mode Functions in pairs, quantifies the coupling relationship between the phase of low-frequency Mode Function and the amplitude of high-frequency Mode Function in each combination, and arranges the quantified coupling relationship to generate a correlation coefficient matrix.

9. The functional network analysis method for complex network according to claim 8, wherein the functional network analysis system further comprises a functional network analysis unit connected to the cross-frequency coupling analysis unit, wherein, the method further comprising:
S4: the functional network analysis unit receives the correlation coefficient matrix sent by the cross-frequency coupling analysis unit; obtain functional network attribute indicators based on Graph theory by using the correlation coefficient matrix as a calculation basis; and presents, in the form of graph, the functional network connection relationship described by the functional network attribute indicators.

10. The functional network analysis method for complex network according to claim 9, wherein the functional network analysis system further comprises a man-machine interaction device connected to the functional network analysis unit, the method further comprising:
S5: the man-machine interaction device displays a man-machine interaction interface to a user; receives a graph, which presents the functional network connection relationship described by functional network attribute indicators, sent by the functional network analysis unit; and displays the graph on the man-machine interaction interface.

11. The functional network analysis method for complex network according to claim 10, wherein the man-machine interaction device is connected to the multi-signal-source measurement unit, the method further comprising:
S0: the man-machine interaction device displays a man-machine interaction interface to a user; acquires a signal source measurement instructions input by the user on the man-machine interaction interface, and sends the signal source measurement instructions to the multi-signal-source measurement unit, wherein the signal source measurement instructions comprise the number of signal sources and the kind of signal sources.

12. The functional network analysis method for complex network according to claim 8, wherein the cross-frequency coupling analysis unit comprises a Mode Function selection module and a correlation coefficient matrix generation module, the method further comprising:
S31: a Mode Function selection module receives the plurality of different Mode Functions, and selects n different Mode Functions from a same signal source or different signal sources; and
S32: a correlation coefficient matrix generation module combines the selected n different Mode Functions in pairs, quantifies coupling relationship between the phase of low-frequency Mode Function and the amplitude of high-frequency Mode Function in each combination, and arrange the quantified coupling relationship to generate C(n,2) m×m correlation coefficient matrixes,
wherein, C(n,2) is obtained by permutation and combination, that is, C(n,2)=n*(n−1)/2, the value of C(n,2) represents the number of the correlation coefficient matrixes; n is the number of the selected different Mode Functions; m is the number of the signal sources; m×m indicates that the number of rows and number of columns of the correlation coefficient matrixes is m; each element in the correlation coefficient matrix is defined as the difference between the probability density distribution of the phase of the j^{th} Mode Function and the amplitude of the i^{th} Mode Function (for short, the probability density distribution of the low-frequency phase and the high-frequency amplitude) and the uniform probability distribution (that is, the probability density distribution of Uniform Distribution), and is used as a measurement indicator for the coupling relationship between the phase of a low-frequency Mode Function and the amplitude of a high-frequency Mode Function from a same signal source or different signal sources, where 1≤i≤n−1, i<j≤n, and j is an integer greater than 1.
